(19) United States
(12) Patent Application Publication
(10) Pub. No.: US 2017/0143256 A1
(43) Pub. Date: May 25, 2017

(54) SYSTEM AND METHOD FOR MEASURING INTENSITY OF PAIN AND EMOTIONAL RESPONSES

(71) Applicant: Annu H. Navani, Fremont, CA (US)

(72) Inventor: Annu H. Navani, Fremont, CA (US)

(21) Appl. No.: 14/949,884

(22) Filed: Nov. 24, 2015

(51) Int. Cl. A61B 5/00; A61B 5/16; A61B 5/01; A61B 5/024; A61B 5/08; A61B 5/145; A61B 5/053; A61B 5/021

(52) U.S. Cl. CPC A61B 5/4824; A61B 5/0022; A61B 5/053; A61B 5/165; A61B 5/021; A61B 5/024; A61B 5/0816; A61B 5/14542; A61B 5/01; A61B 5/7275; A61B 5/4848; A61B 5/6826

(57) ABSTRACT

A system for determining at least one medical condition in a user includes an external communication device, and a portable device for measuring intensity of pain and multiple emotional responses of a user, and generating data associated with skin conductance of the user. This data is used independently or integrated with other measurements. Predictive algorithms can be established for evaluation and treatment of the individual. Particularly, the portable device is capable of communicating with the external communication device over a network.

### BACKGROUND OF THE INVENTION

Field of the Invention

Embodiments of the present invention generally relate to detecting one or more medical conditions, and more particularly, to a system and method for detecting medical conditions in a user by measuring intensity of pain and multiple emotional responses.

Description of the Related Art

Over the years, medical treatment of acute injuries and illnesses have improved remarkably. However, chronic disease remains by far the greatest cause of mortality, diminished quality of life of individuals, and increased healthcare expenditures. Currently, approximately 80% of healthcare costs are spent on the treatment of chronic disease, much of it on unnecessary hospitalizations, inappropriate medical interventions, and poor overall coordination of care. This is true because chronic diseases are commonly initially treated but quite frequently not appropriately managed in the long term. Moreover, chronic pain is a very common condition that has a huge impact in well being and economy of any area. Chronic pain refers to severe and distressing pain that may interfere with daily life and that continues for six months or more. This type of pain is called “persistent somatoform pain disorder” by the International Statistical Classification of Diseases and Related Health Problems, 10th Revision (ICD-10). It is suggested that psychological factors have a major impact on the onset and exacerbation of chronic pain; however, its cause remains unknown.

Currently, 100 million Americans suffer from chronic pain. This has lead to loss of productive time of individuals who are suffering from common painful conditions and it has been estimated to be $61.2 billion per year, while 76.6% of lost productive time was explained by reduced work performance, and not absenteeism. It is a well known fact, that prescription drugs are the second-most abused category of drugs in the United States, following marijuana. While 43% of pain sufferers have been to only one type of doctor for their pain illness, a large proportion (38%) have consulted more than one practitioner in the medical community. Seven in ten Americans feel that pain research and management should be one of the medical community's top few priorities (16%) or a high priority (55%). Almost six in 10 adults (57%) say that they would be willing to pay one dollar more per week in taxes to increase federal funding for the scientific research and development into the causes and treatment of pain.

Further, anxiety disorders are the most common mental illness in the U.S., affecting 40 million adults in the United States age 18 and older and that is approximately 18% of U.S. population. Anxiety disorders cost the U.S. more than $42 billion a year, almost one-third of the country's $148 billion total mental health bill, according to “The Economic Burden of Anxiety Disorders,” a study commissioned by ADAA (The Journal of Clinical Psychiatry, 60(7), July 1999). Depression, adjustment disorder and other psychiatric conditions either independently or intertwined with chronic pain are also huge burden to the growth of the economy.

Abuse of tobacco, alcohol, and illicit drugs is costly to any Nation, exacting over $600 billion annually in costs related to crime, lost work productivity and healthcare. Alcoholism and alcohol abuse are among the most common, devastating, and costly problems in the United States. In fact, recent studies have shown that approximately 53 percent of adults in the United States have reported that one or more of their close relatives are suffering from a drinking problem. U.S. alcohol statistics reveal that approximately 50,000 cases of alcohol overdose are reported each year. In 2009, an estimated 30.2 million people 12 or older reported driving under the influence of alcohol at least once in the past year.

Conventionally, there is no objective way to measure pain in a clinical setting. In current scenario, pain is measured by patient reported numerical or verbal scores. However, this is very subjective and unreliable. Moreover, the degree of pain (pain index) the patient feels has been traditionally measured through pencil-and paper methods for example, subjective questionnaires. In a demented or critically ill patient or in neonates and infants where communication is not possible, the vital signs are used by clinician to measure pain but those have been noted to be unreliable measures of pain to provide a proper diagnosis.

Currently, the degree of pain the patient feels can be measured through bio-measures for example, skin sweat, pulse, respiration rhythm or volume. There are few devices available that measure skin conductance of the patient. First essence that is cumbersome skin electrodes and second neumitra watch which is a wearable device. The wearable device is very expensive. Both relate to skin conductance readings to emotional well being and not pain. Moreover, the already existing wearable devices in this field suffer from manufacturing and distribution challenges. Additionally, they are limited in functionality by their dedicated hardware design. Furthermore, dedicated devices may not already be owned by the patient, requiring them to acquire such a device at additional cost, availability and inconvenience. As a result, the current market still lacks an affordable and effective way to measure a patient for their mental or physical state without a face-to-face meeting or at best a phone based interview.

Particularly, the above listed measures are subjective and can be effected significantly by confounding factors related to the subject and observer. Therefore, there remains a urgent need for a device that is able to objectively measure pain and emotional responses, and at the same time has the ability to recommend treatments based on the data collected in real time. The data collected and interpreted can include raw measurement data from the device or a revised version after integration and correlation with other measurement tools such as self reported pain level, standardized psychiatric tests, vital signs such as heart rate, blood pressure, respiration, oxygen level, sympathetic response and the like. The recommended treatments generated by the present device and system includes self guided tools that provides patient with the tool to manage their pain and emotions.

Accordingly, there remains a need to develop systems and methods to assist patients score their pain objectively and get access to independent treatment without waiting for doctor's appointment or visit. The present system is able to measure pain and level of psychiatric impact on subjects that has the potential be used to screen for continuity of care, disability predictions, treat pain in patients who are unable to communicate such as neonates and infants, demented or comatosed patients or patients with language barriers.

### SUMMARY OF THE INVENTION

Embodiments of the present disclosure provide a system for determining at least one medical condition in a user by measuring intensity of pain and multiple emotional responses in body of the user. In one embodiment, the system includes, an external communication device and a portable device for measuring intensity of pain and multiple emotional responses of a user, and generating data associated with skin conductance of the user and the portable device being capable of communicating with the external communication device over a network. Particularly, the external communication device includes an interface operative to provide multiple graphical icons to the user. In operation, the portable device includes at least one bio-sensor configured to sense at least one body signal from the user, a data recorder connected to the at least one bio-sensor for recording data associated with the at least one body signal of the user, and, a communicator connected to the data recorder for transmitting over the network, the data associated with the at least one body signal of the user to one or more external communication devices.

In accordance with one embodiment of the present invention, the data associated with the at least one body signal of the user is retrieved at multiple time intervals.

In accordance with one embodiment of the present invention, the one or more bio-sensors include multiple skin conductance measurement electrodes and the bio-sensor is capable of contacting skin of the user for retrieving skin conductance associated data of the user.

In accordance with one embodiment of the present invention, the external communication device further includes, at least one central processor configured to execute multiple instructions, and includes the steps of storing the skin conductance associated data of the user measured at multiple time intervals in a database; processing and analyzing the skin conductance associated data to determine the at least one medical condition by retrieving the data from the database; tracking progress of treatment of the user by processing the data associated with skin conductance at multiple time intervals; and determining effectiveness of the self-guided therapy recommendation for treating the at least one medical condition in the user.

Embodiments of the present disclosure further provide a method for determining at least one medical condition in a user by measuring intensity of pain and multiple emotional responses in body of the user. The method includes the steps of measuring skin conductance of a user at multiple time intervals, recording data associated with skin conductance at multiple time intervals, processing the data to generate a skin conductance score of the user to determine at least one medical condition, integrating the data with other relevant measures such as standardized scales, vital signs and the like, generating self-guided therapy recommendation for treating the at least one medical condition, providing the self-guided therapy recommendation for treating the at least one medical condition in the user; and transmitting and storing the data associated with skin conductance to at least one external communication device.

Embodiments of the present disclosure further provides, a portable device for measuring intensity of pain and multiple emotional responses of a user. The portable device includes at least one bio-sensor configured to sense at least one body signal from the user, a data recorder connected to the at least one bio-sensor for recording data associated with the body signal of the user, and a communicator connected to the data recorder for transmitting over a network, the data associated with the at least one body signal of the user to at least one external communication device. Specifically, the data associated with the at least one body signal of the user is retrieved at multiple time intervals.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a block diagram illustrating a system for determining medical condition in a user and the portable device configured for measuring intensity of pain and multiple emotional responses of the user, in accordance with one embodiment of the present invention;

FIG. 2 illustrates a pictorial representation of one or more bio-sensors of the portable device for measuring intensity of pain and multiple emotional responses of the user, in accordance with one embodiment of the present invention;

FIG. 3 illustrates a block diagram of a system for determining at least one medical condition in a user by measuring intensity of pain and multiple emotional responses in body of the user, in accordance with one embodiment of the present invention; and

FIG. 4 illustrates a flowchart of a method for determining at least one medical condition in a user by measuring intensity of pain and multiple emotional responses in body of the user, in accordance with one embodiment of the present invention;

While the present systems and methods have been described herein by way of example for several embodiments and illustrative drawings, those skilled in the art will recognize that the multiple embodiments disclosed hereinbelow are not limited to the embodiments or drawings described. It should be understood, that the drawings and detailed description thereto are not intended to limit embodiments to the particular form disclosed. Rather, the intention is to cover all modifications, equivalents and alternatives falling within the spirit and scope of the invention as defined by the appended claims. Any headings used herein are for organizational purposes only and are not meant to limit the scope of the description or the claims. As used herein, the word “can” and “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION

Various embodiments of the present invention relate to systems and methods for real time collection of patient data, analyzing the data and providing self-guided recommendations to the users. The users are able to score their pain and psychiatric/emotional level objectively and get access to preliminary treatment without waiting for doctor's appointment or visit. Specifically, the embodiments of the present invention are aimed at providing an innovative application embodying systems and methods as described herein below for providing the user the platform to input information regarding the user health progress, goal attainment and additional analysis of data by the present device which leads to set of recommendations for the user to follow and the cycle repeats until the user goal is achieved. In use, the one or more embodiments of present invention provide an automated real time approach, which measures skin conductance data of the user, and the present system and method interpret the data to score pain and recommend therapies based on many parameters. Consequently, such innovative features embodied using various aspects of the present invention provide a real-time platform to the user to input necessary data fields, follow automatic recommended guidelines, track progress by entering data, and follow recommended guidelines until user goal is met.

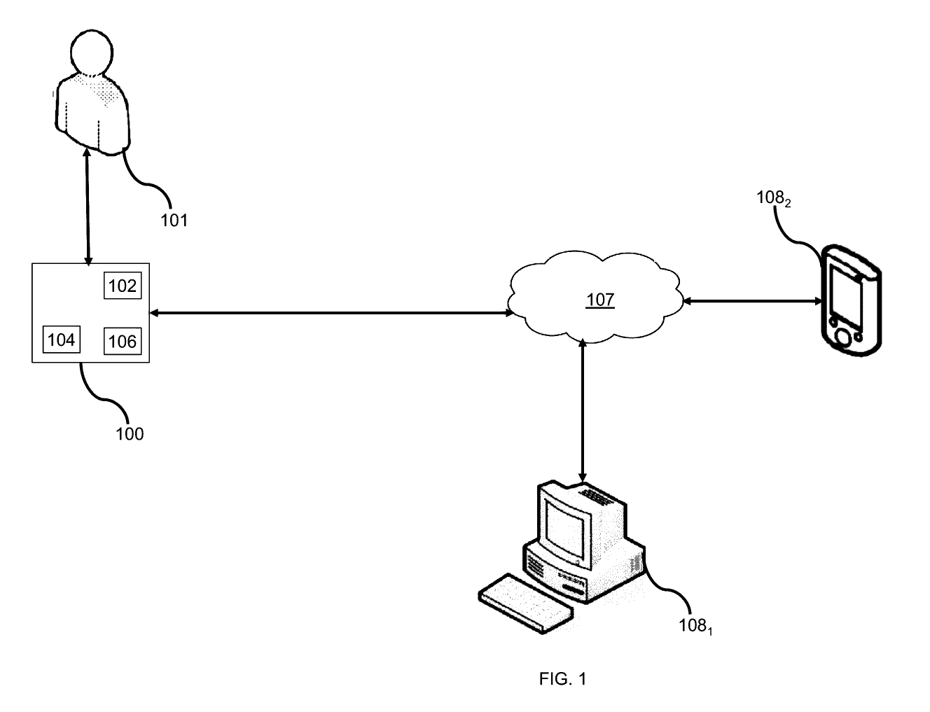

FIG. 1 illustrates a block diagram illustrating a system for determining medical condition in a user and the portable device 100 configured for measuring intensity of pain and multiple emotional responses of the user. In accordance with an embodiment of the present invention, the portable device 100 for measuring intensity of pain and multiple emotional responses of the user includes, one or more bio-sensors 102 configured to sense one or more body signals from the user 101, a data recorder 104 connected to the bio-sensors 102 for recording data associated with the body signals of the user 101, and, a communicator 106 connected to the data recorder 104 for transmitting over a network 107, the data associated with the body signals of the user 101 to one or more external communication devices 108 (108₁, 108₂). The communicator 106 is also capable of providing for wireless and wired communications between various devices and the portable device 100. These communication sessions may involve network communications or wireless communications, such as WI-FI, Bluetooth, RF, or other type of wireless communication. In use, the portable device 100 is a wearable device.

In addition, the external communication device 108₁ is an external computer or the external communication device 108₂ is an external smartphone. In accordance with an embodiment of the present invention, the external communication device 108 is a handheld device, such as, for example, but not limited to, a smartphone, a tablet, a laptop, and the like.

In accordance with an embodiment of the present invention, the body signals of the user 101 are retrieved at multiple time intervals. Generally, auto regulation of the skin temperature influences the skin conductance, which is correlated with skin temperature of human body. Accordingly, retrieving the body signals of the user 101 at multiple time intervals may reveal significant changes in one or more vital parameters, thereby increasing sensitivity and specificity for the measurement of pain.

In accordance with another embodiment of the present invention, the one or more bio-sensors 102 are externally connected to the portable device 100 for measuring intensity of pain and multiple emotional responses of the user.

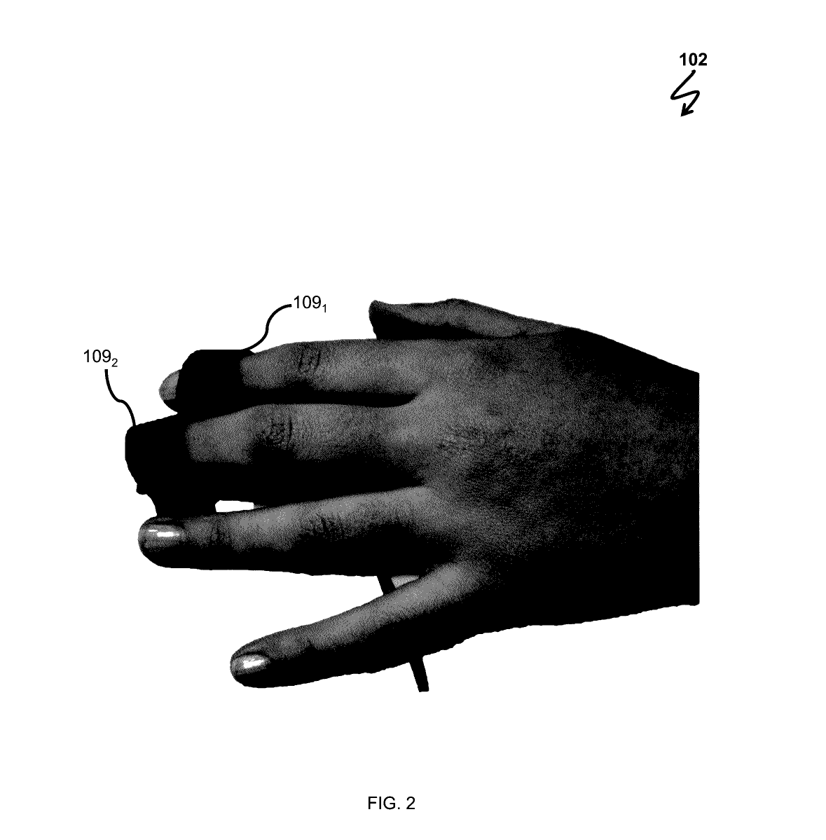

FIG. 2 illustrates a pictorial representation of the bio-sensors 102 of the portable device 100 for measuring intensity of pain and multiple emotional responses of the user 101. In accordance with an embodiment of the present invention, the bio-sensor 102 includes multiple skin conductance measurement electrodes 109 (109₁, 109₂). Those of ordinary skill in the art will appreciate that the number of electrodes 109 may vary depending upon exact need. Consequently, the portable device 100 includes one or more bio-sensors 102, which can further include skin multiple conductance measurement electrodes 109 (109₁, 109₂) to measure skin conductance of the user 101 across various body parts of the user 101. Those of ordinary skill in the art will further appreciate that in FIG. 2, hand of user 101 is illustrated for reference purposes only and various elements of the present invention may be configured to be utilized for multiple body parts of the user 101. Moreover, the bio-sensor 102 is capable of contacting skin of the user for retrieving skin conductance associated data of the user 101.

In accordance with another embodiment of the present invention, the one or more bio-sensors 102 which includes skin multiple conductance measurement electrodes 109 (109₁, 109₂) to measure skin conductance of the user 101 is employed as an external attachment to the portable device 100. The one or more bio-sensors 102 are applied an attachment and is electrically connected to the portable device 100 to measure skin conductance and record the reading on the device 100.

In accordance with an embodiment of the present invention, the data recorder 104 is capable of recording the data associated with skin conductance of the user 101 retrieved at multiple time intervals. Additionally, the communicator 106 is capable of transmitting over the network 107, the data associated with skin conductance of the user 101 to the external communication devices 108 at multiple time intervals. Subsequently, such data that is recorded and transmitted to the external communication devices 108 at multiple time intervals may be used for measuring intensity of pain and multiple emotional responses of the user 101. It will be appreciated that the multiple time intervals may depend upon multiple parameters, such as, for example, but not limited to, age of the user, medical condition of the user, weight of the user, body mass index (BMI) of the user, and the like.

Those of ordinary skill in the art will appreciate that the one or more body signals may include, at least one of, an autonomic signal selected from a blood pressure, a pulse rate, a respiratory rate, a blood oxygen saturation, or a body temperature; a pain signal; and an emotional signal selected from depression, anxiety, fear, claustrophobia, stress, insomnia, anger, irritability and any other mental state of the user 101. In use, the one or more body signals may be correlated with the one or more bio-sensors 102 for additional data.

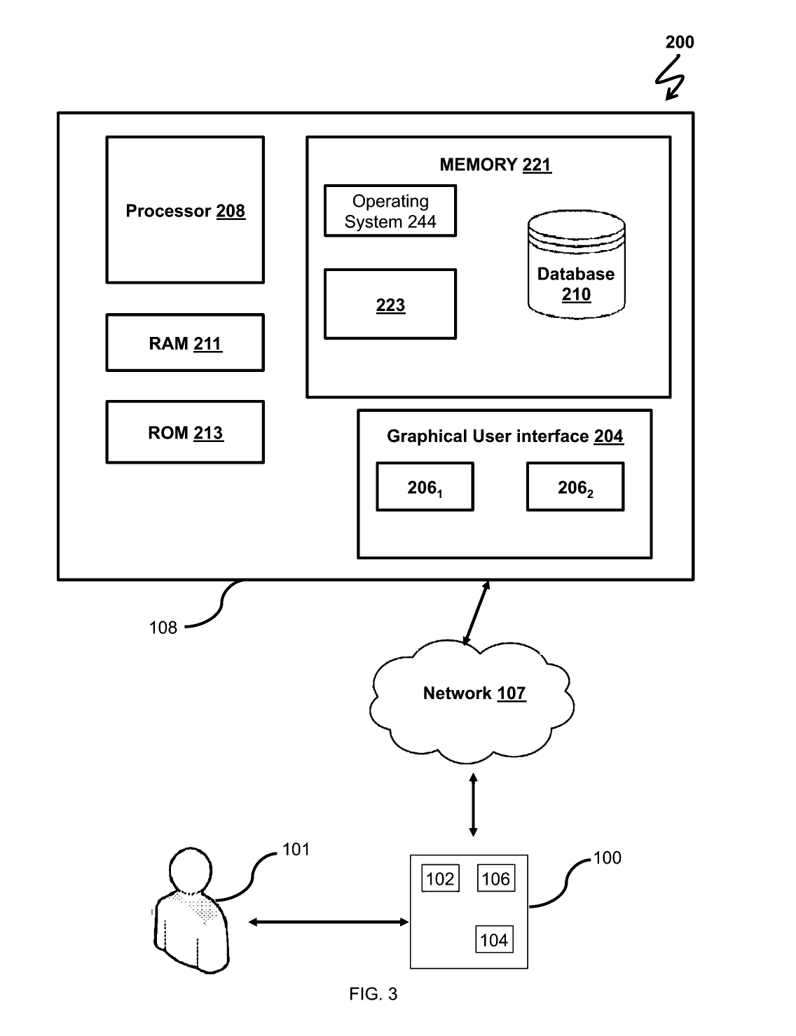

FIG. 3 illustrates a block diagram of a system 200 for determining one or more medical conditions in the user 101 by measuring intensity of pain and multiple emotional responses in body of the user 101. In accordance with an embodiment of the present invention, the system 200 includes, an external communication device 108 including an interface 204 operative to provide multiple graphical icons 206 (206₁, 206₂) to the user 101, and, the portable device 100 for measuring intensity of pain and multiple emotional responses of the user 101, and generating data associated with skin conductance of the user 101.

In accordance with an embodiment of the present invention, the external communication device 108 further includes, one or more central processors 208 configured to execute multiple instructions, including, storing the skin conductance associated data of the user 101 measured at multiple time intervals in a database 210, processing and analyzing the skin conductance associated data to determine the medical conditions by retrieving the data from the database 210, tracking progress of treatment of the user 101 by processing the data associated with skin conductance at the time intervals, and, determining effectiveness of the self-guided therapy recommendation for treating the medical conditions in the user 101.

In accordance with an embodiment of the present invention, the central processor 208 is further configured to execute multiple instructions, including, modifying and recommending a different therapy based on the skin conductance score of the user 101 measured at different time intervals. In use, the central processor 208 is configured to execute multiple instructions to correlate a certain score to a certain level of pain and recommend self-guided therapy accordingly. Specifically, the central processor 208 is configured to execute multiple instructions, including, all the steps of method 400. In use, the graphical icons 206 (206₁, 206₂, 206₃, 206₄, . . . 206ₙ) may be configured as per need to embody various systems and methods as disclosed herein.

Those of ordinary skill in the art will appreciate that the external communication device 108 may be similar to any available computing device, such as a personal computer (e.g., a desktop computer), server, laptop computer, notebook, tablet, smartphone, etc. Various elements of the external communication device 108 as explained above may be implemented with one or more processors 208 and one or more storage units (e.g., databases, RAM, ROM, and other computer-readable media), one or more application specific integrated circuits (ASICs), and/or other hardware components.

It will be further appreciated that the processor 208 may be capable of controlling operations of the external communication device 108 and its associated components, including RAM 211, ROM 213, the graphical user interface 204, and memory 221. The memory 221 may be any computer readable medium for storing computer executable instructions (e.g., software). The instructions stored within memory 221 may enable the system 200 to perform various functions. For example, memory 221 may store software used by the external communication device 108, such as an operating system 244 and application programs 223, and may include a database 210. The graphical user interface 204 allows the external communication device 108 to connect to and communicate with the network 107. The network 107 may be any type of network, including a local area network (LAN) and/or a wide area network (WAN), such as the Internet, a cellular network, a satellite network or other platforms. The network 107 may operate in accordance with one or more communication standards, such as standards promulgated by the Institute of Electrical and Electronics Engineers (IEEE) (e.g., an IEEE 802.11 standard). Through the network 107, the external communication device 108 may communicate with one or more portable devices 100. In some embodiments, the external communication device 108 may be connected to the portable device 100 to form a “cloud” computing environment.

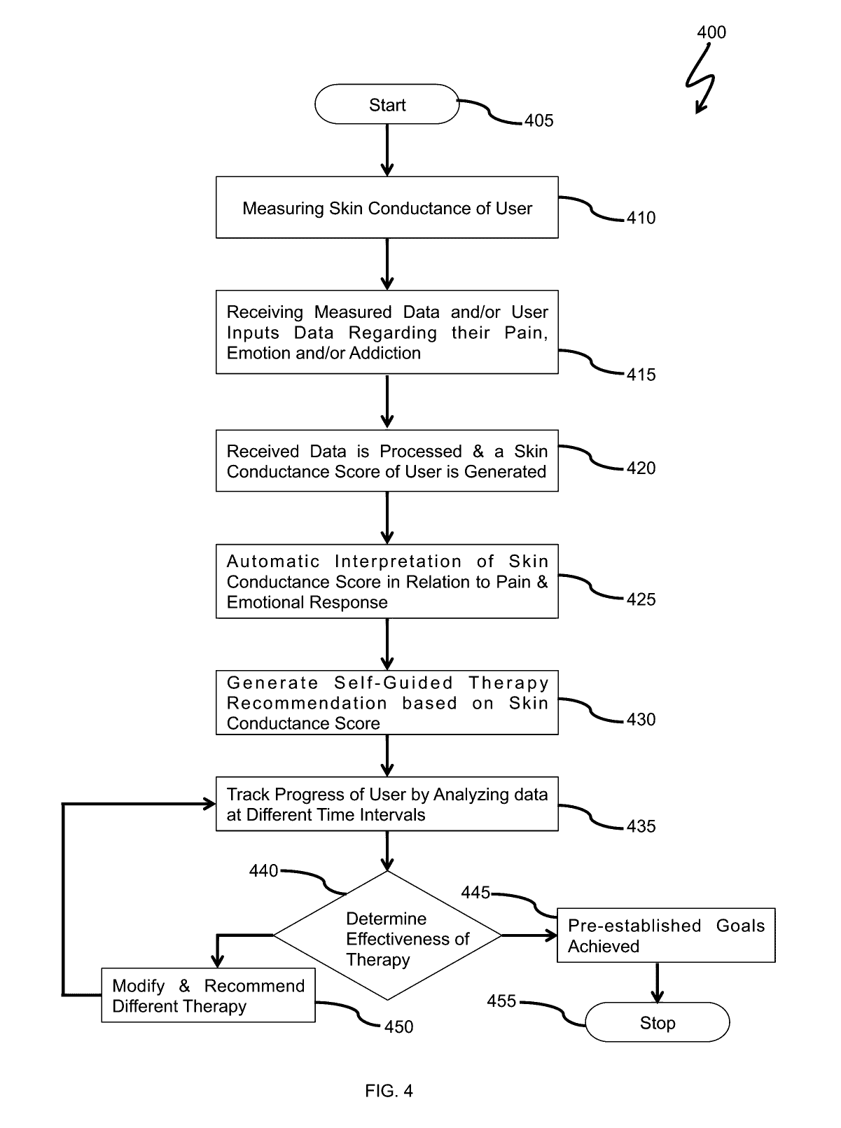

FIG. 4 illustrates a flowchart of method 400 for determining at least one medical condition in a user by measuring intensity of pain and multiple emotional responses in body of the user, in accordance with one embodiment of the present invention. The method 400 begins at step 405 and proceeds to step 410. At step 410, the method 400 includes, measuring skin conductance of the user 101 via the portable device 100.

In accordance with an embodiment of the present invention, the skin conductance of the user 101 is measured at multiple time intervals. The method 400 then proceeds to step 415. At step 415, the method 400 includes the step of recording the data associated with skin conductance at multiple time intervals by the data recorder 104 of the portable device 100.

In accordance with another embodiment of the present invention, the user 101 inputs manual data associated with one or more parameters of pain, emotion and addiction to medications, drugs, substances and/or any particular substance, thing, or activity. The details of emotion parameters of the user include mental state of the user. For example, emotional state of depression, anxiety, fear, claustrophobia and any other known mental states. The recorded data associated with the body signals of the user 101 is transmitted over the network 107 by the communicator 106 to one or more external communication devices 108 (108₁, 108₂). The method 400 then proceeds to step 420. At step 420, the received data is processed by the one or more external communication devices 108 to generate a skin conductance score of the user to determine the medical condition of the user. In use, the processor 208 of the external communication device 108 is configured to execute multiple instructions to correlate a certain score to a certain level of pain by retrieving the skin conductance associated data of the user 101 from the database 210.

The method 400 then proceeds to step 425. At step 425, the method 400 includes the step of automatic interpretation of skin conductance score in relation to pain & emotional response by the processor 208 of the external communication device 108. The instructions executed by the processor 208 confirms the patient reported score of pain and validate that subjective score is individualized to the user 101. Therefore, the data is interpreted in terms of clinical relevance.

The method 400 then proceeds to step 430. At step 430, the processor 208 executes instructions to generate a self-guided therapy recommendation for treating the at least one medical condition based on the skin conductance score of the user 101. The generated self-guided therapy recommendation is communicated to the external communication device 108₂ via the network 107 to the user 101.

The method 400 then proceeds to step 435. At step 435, progress of treatment of the user 101 is tracked by processing the data associated with skin conductance recorded at the different time intervals by the portable device 100.

In another embodiment, the method 400 further includes the step of receiving user input data by the user 101 at different time intervals by the portable device 100. Particularly, the data includes information regarding pain, emotion and addiction.

The method 400 then proceeds to step 440. At step 440, a determination is made to determine effectiveness of the self-guided therapy recommendation for treating the at least one medical condition associated with the user 101. If the answer is “YES”, and the self-guided therapy recommendation is effective the method 400 proceeds to step 445. At step 445, the pre-established goals for the user 101 are achieved and self-guided therapy recommendation is useful to cure the ailment. The method 400 then proceeds to step 455. At step 455, the method 400 ends.

In another embodiment, if the answer is “NO”, and it is determined that the self-guided therapy recommendation for treating the at least one medical condition in the user 101 is not effective the method 400 proceeds to step 450. At step 450, the method 400 further includes the step of modifying and recommending a different therapy based on the skin conductance score of the user 101. The method 400 then proceeds to step 435.

Therefore, as may be seen, various embodiments of the present invention disclose systems and methods to collect user data by user filling in necessary data fields, following automatic recommended guidelines, tracking progress by entering data, and following recommended guidelines until user goal is met. The present invention assists the users to score their pain objectively and get access to preliminary treatment while waiting for doctor's appointment or visit. Moreover, the present system and method empower patients to take control of their treatments, feel committed and self-guide their treatments. Therefore, the present invention will decrease health care costs, improve outcomes and long term adherence. It is affordable, accurate and user friendly.

The present system and the portable device will measure pain in additional to emotional responses. The present system provides a universal objective way to measure pain that could be potentially be used to screen disability eligibility due to chronic pain, treat pain associated with injuries, manage post surgical and all other types of pain and also gauge and treat pain and emotional responses in patients who are unable to communicate such as neonates and infants, demented or comatosed patients or patients with language barriers.

The present method executes a set of instructions to measure the intensity of pain and emotional responses in human body using skin conductance and use of it to provide self-guided therapies for those conditions in real time to the patients. Particularly, the measurement of skin conductance of the user provides objective measure of human and possibly animal emotional reactions such as depression, anxiety, stress, insomnia, anger and irritability. Subsequently, the present method is configured to record pain, all types of emotions or mental states for example, depression, anxiety, fear, claustrophobia and other mental states and addiction to medications, drugs, substances and/or any particular substance, thing, or activity and provide a non chemical self-guided individualized therapies to address the above mentioned conditions. Subsequently, the present invention will empower subjects to take control of their treatments, and this will eventually decrease healthcare costs and improve outcomes and long term adherence.

As may be seen from above-mentioned description of various embodiments, the users are provided with a simple and interactive application on their handheld devices (such as, a smart phone) and with just a few interactions with graphical user icons, the users are empowered to take control of their treatments, feel committed and self-guide their treatments, and thereby decreasing healthcare costs and improve outcomes and long term adherence. In operation, the present method executes a set of instructions to use and interpret data entered and/or skin conductance in relation to pain and emotional reactions.

Moreover, as compared to previously available solutions to patients, the embodiments of the present invention provide significant improvements and advantages by providing the users (patients) to keep track of their progress by analyzing their self-reported outcomes, which can be accessed through the smartphone and modify recommendations based on their progress monitored by the portable device. Therefore, as may be seen, embodiments of the present invention provide an integrated, expeditious and convenient solution accessible from user's smart phone. In addition, the applications that embody various aspects of the present invention are accomplished by collecting, storing, analyzing the data and adapting different recommendations based on the data collected and made available to the users.

Accordingly, while there has been shown and described the preferred embodiment of the invention is to be appreciated that the invention may be embodied otherwise than is herein specifically shown and described and, within said embodiment, certain changes may be made in the form and arrangement of the parts without departing from the underlying ideas or principles of this invention within the scope of the claims appended herewith.

What is claimed is:

1. A portable device for measuring intensity of pain and a plurality of emotional responses of a user, said portable device comprising:
at least one bio-sensor configured to sense at least one body signal from said user;
a data recorder connected to said at least one bio-sensor for recording data associated with said at least one body signal of said user; and,
a communicator connected to said data recorder for transmitting over a network, said data associated with said at least one body signal of said user to at least one external communication device,
wherein said data associated with said at least one body signal of said user is retrieved at a plurality of time intervals.

2. The portable device as claimed in claim 1, wherein said at least one bio-sensor comprises a plurality of skin conductance measurement electrodes and wherein said at least one bio-sensor is capable of contacting skin of said user for retrieving skin conductance associated data of said user.

3. The portable device as claimed in claim 2, wherein said data recorder is capable of recording said data associated with skin conductance of said user retrieved at said plurality of time intervals.

4. The portable device as claimed in claim 2, wherein said communicator is capable of transmitting over said network, said data associated with skin conductance of said user to said at least one external communication device at said plurality of time intervals.

5. The portable device as claimed in claim 1, wherein said plurality of time intervals depends upon a plurality of parameters.

6. The portable device as claimed in claim 5, wherein said plurality of parameters are selected from a group comprising at least one of, age of said user, medical condition of said user, weight of said user, body mass index (BMI) of said user, and the like.

7. The portable device as claimed in claim 1, wherein said at least one body signal is at least one of: an autonomic signal selected from a blood pressure, a pulse rate, a respiratory rate, a blood oxygen saturation, or a body temperature; a pain signal; and an emotional signal selected from depression, anxiety, fear, claustrophobia, stress, insomnia, anger, irritability and any other mental state of said user.

8. A system for determining at least one medical condition in a user by measuring intensity of pain and a plurality of emotional responses in body of said user, said system comprising:
an external communication device comprising an interface operative to provide a plurality graphical icons to said user; and
a portable device for measuring intensity of pain and a plurality of emotional responses of a user, and generating data associated with skin conductance of said user, and said portable device being capable of communicating with said external communication device over a network, said portable device comprising:
at least one bio-sensor configured to sense at least one body signal from said user;
a data recorder connected to said at least one bio-sensor for recording data associated with said at least one body signal of said user; and,
a communicator connected to said data recorder for transmitting over said network, said data associated with said at least one body signal of said user to at least one external communication device,
wherein said data associated with said at least one body signal of said user is retrieved at a plurality of time intervals.

9. The system as claimed in claim 8, wherein said at least one bio-sensor comprises a plurality of skin conductance measurement electrodes and wherein said at least one bio-sensor is capable of contacting skin of said user for retrieving skin conductance associated data of said user.

10. The system as claimed in claim 9, wherein said external communication device further comprises, at least one central processor configured to execute a plurality of instructions, comprising:
storing said skin conductance associated data of said user measured at said plurality of time intervals in a database;
processing and analyzing said skin conductance associated data to determine said at least one medical condition by retrieving said data from said database;
tracking progress of treatment of said user by processing said data associated with skin conductance at said plurality of time intervals; and
determining effectiveness of said self-guided therapy recommendation for treating said at least one medical condition in said user.

11. The system as claimed in claim 10, wherein said at least one central processor is further configured to execute a plurality of instructions, comprising:
modifying and recommending a different therapy based on said skin conductance score of said user.

12. The system as claimed in claim 8, wherein said data recorder is capable of recording said skin conductance associated data of said user retrieved at said plurality of time intervals.

13. The system as claimed in claim 8, wherein said communicator is capable of transmitting over said network, said skin conductance associated data of said user to said external communication device at said plurality of time intervals.

14. The system as claimed in claim 8, wherein said plurality of time intervals depends upon a plurality of parameters.

15. The system as claimed in claim 14, wherein said plurality of parameters are selected from a group comprising at least one of, age of said user, medical condition of said user, weight of said user, body mass index (BMI) of said user, and the like.

16. The system as claimed in claim 8, wherein said at least one body signal is at least one of: an autonomic signal selected from a blood pressure, a pulse rate, a respiratory rate, a blood oxygen saturation, or a body temperature; a pain signal; and an emotional signal selected from depression, anxiety, fear, claustrophobia, stress, insomnia, anger, irritability and any other mental state of said user.

17. A method for determining at least one medical condition in a user by measuring intensity of pain and a plurality of emotional responses in body of said user, said method comprising the steps of:
measuring skin conductance of a user at a plurality of time intervals;
recording data associated with skin conductance at said plurality of time intervals;
processing said data to generate a skin conductance score and correlating with other measurements of said user to determine at least one medical condition;
generating self-guided therapy recommendation for treating said at least one medical condition;
providing said self-guided therapy recommendation for treating said at least one medical condition in said user; and
transmitting and storing said data associated with skin conductance to at least one external communication device.

18. The method as claimed in claim 17, wherein said method further comprises the steps of:
tracking progress of treatment of said user by processing said data associated with skin conductance at said plurality of time intervals; and
determining effectiveness of said self-guided therapy recommendation for treating said at least one medical condition in said user.

19. The method as claimed in claim 17, wherein said method further comprises the step of receiving user input data by said user and wherein said data comprises information regarding pain, emotion and addiction.

20. The method as claimed in claim 18, wherein said method further comprises the step of modifying and recommending a different therapy based on said skin conductance score of said user.
